(19) United States
(12) Patent Application Publication
(10) Pub. No.: US 2011/0230668 A1
(43) Pub. Date: Sep. 22, 2011

(54) CATALYST FOR GAS PHASE OXIDATIONS BASED ON LOW-SULFUR AND LOW-CALCIUM TITANIUM DIOXIDE

(75) Inventors: Stefan Altwasser, Wachenheim (DE); Christine Deißler, Mannheim (DE); Andrey Karpov, Mannheim (DE); Cornelia Katharina Dobner, Ludwigshafen (DE); Bastian Ewald, Ludwigshafen (DE); Frank Rosowski, Mannheim (DE); Hagen Wilmer, Ludwigshafen (DE)

(73) Assignee: BASF SE, Ludwigshafen (DE)

(21) Appl. No.: 13/052,140

(22) Filed: Mar. 21, 2011

Related U.S. Application Data
(60) Provisional application No. 61/315,463, filed on Mar. 19, 2010.

(51) Int. Cl. C07D 307/89; B01J 27/02; B01J 27/198

(52) U.S. Cl. 549/248; 502/216; 502/209

(57) ABSTRACT

A catalyst for gas phase oxidations comprises an inert support and a catalytically active material which comprises vanadium oxide and titanium dioxide and has been applied thereto. The titanium dioxide has a content of sulfur compounds, calculated as S, of less than 1000 ppm and a content of calcium compounds, calculated as Ca, of less than 150 ppm. The catalyst has a relatively high activity and/or selectivity and thus enables relatively high yields of the desired target product, for example phthalic anhydride. Also described is a process for preparing phthalic anhydride, wherein a gas stream which comprises molecular oxygen and o-xylene, naphthalene or mixtures thereof is contacted with the catalyst.

The invention relates to a catalyst for gas phase oxidations, which comprises an inert support and a catalytically active material which comprises vanadium oxide and titanium dioxide and has been applied thereto, to a process for preparation thereof and to the use of the catalyst for preparing phthalic anhydride.

A multitude of carboxylic acids and/or carboxylic anhydrides is prepared industrially by the catalytic gas phase oxidation of aromatic hydrocarbons, such as benzene, the xylenes, naphthalene, toluene or durene, in fixed bed reactors. In this way, it is possible to obtain, for example, benzoic acid, maleic anhydride, phthalic anhydride (PSA), isophthalic acid, terephthalic acid or pyromellitic anhydride. In general, a mixture of an oxygenous gas and the starting material to be oxidized is passed through tubes in which there is a bed of a catalyst. To regulate the temperature, the tubes are surrounded by a heat carrier medium, for example a salt melt.

Useful catalysts for these oxidation reactions have been found to be so-called eggshell catalysts, in which the catalytically active material has been applied in the form of a shell on an inert support material such as steatite. The catalytically active constituent of the catalytically active material of these eggshell catalysts is generally, in addition to titanium dioxide, vanadium pentoxide. In addition, a multitude of other oxidic compounds which influence the activity and selectivity of the catalyst as promoters may be present in small amounts in the catalytically active material.

The influence of impurities in the titanium dioxide used has been examined in the prior art. Grzybowska-Swierkosz mentions, in Appl. Catal. A: Gen. 157 (1997) 263-310, that impurities on the surface of commercial titanium dioxide pigments can influence the structure of surface-bound OH groups.

WO 2007/134849 describes the use of particular titanium dioxides for preparing a catalyst for oxidation of o-xylene to phthalic anhydride. The titanium dioxide should comprise less than 1000 ppm of sulfur, less than 300 ppm of phosphorus and more than 500 ppm of niobium.

Garcin et al. (Catalysis Today 20 (1994) 7-10) utilizes a titanium dioxide for a PSA catalyst which is characterized by the following impurities: 0.12% by weight of sulfate, <0.005% by weight of SiO₂, <0.005% by weight of Al₂O₃, 0.04% by weight of K₂O, <0.002% by weight of Sb₂O₃, <0.22% by weight of Nb₂O₅, 0.02% by weight of ZrO₂, <0.002% by weight of SnO₂, 27 ppm of Fe, 0.24% by weight of P₂O₅ and 0.023% by weight of CaO (corresponding to 164 ppm of Ca).

EP-A 539878 states that the secondary Fe, Zn, Al, Mn, Cr, Ca, and Pb components in the titanium dioxide are not disruptive, provided that the total amount thereof is not more than 0.5% by weight (as the metal oxide), based on the amount of titanium dioxide.

There is a constant need for catalysts for gas phase oxidations which have a maximum conversion coupled with high selectivity.

It is an object of the invention to specify a catalyst for gas phase oxidations which has a relatively high activity and/or selectivity and thus enables relatively high yields of the desired target product, for example phthalic anhydride.

The object is achieved by a catalyst for gas phase oxidations, comprising an inert support and a catalytically active material which comprises vanadium oxide and titanium dioxide and has been applied thereto, wherein the titanium dioxide has a content of sulfur compounds, calculated as S, of less than 1000 ppm and a content of calcium compounds, calculated as Ca, of less than 150 ppm.

The invention also relates to a process for preparing a catalyst for gas phase oxidations, in which a suspension of titanium dioxide and vanadium oxide particles is applied to an inert support, wherein the titanium dioxide has a content of sulfur compounds, calculated as S, of less than 1000 ppm and a content of calcium compounds, calculated as Ca, of less than 150 ppm. In a preferred embodiment, the application of at least a portion of the titanium dioxide is preceded by treatment with an aqueous medium under hydrothermal conditions.

The titanium dioxide used in accordance with the invention has a particular content of sulfur compounds and calcium compounds. The chemical impurities of the TiO₂, more particularly the S, Ca, P and Nb contents, are determined to DIN ISO 9964-3. This involves determining the contents by means of ICP-AES (Atomic Emission Spectroscopy with Inductively Coupled Plasma).

When mixtures of different titanium dioxides are used, the content of sulfur compounds and calcium compounds is determined as the weighted mean of the contents of the individual titanium dioxides in the mixture. The use of mixtures of different titanium dioxides may be suitable, for example, for establishing a desired value of the BET surface area, by mixing a titanium dioxide of high BET surface area and a titanium dioxide of low BET surface area in particular proportions.

In preferred embodiments, the titanium dioxide has a content of sulfur compounds of less than 500 ppm, especially less than 400 ppm, for example 100 to 300 ppm.

In preferred embodiments, the titanium dioxide has a content of calcium compounds, calculated as Ca, of less than 100 ppm, especially less than 80 ppm, for example 50 to 75 ppm.

In preferred embodiments, the titanium dioxide additionally has a content of phosphorus compounds, calculated as P, of less than 1000 ppm, especially less than 500 ppm, for example 100 to 300 ppm.

In preferred embodiments, the titanium dioxide additionally has a content of niobium compounds, calculated as Nb, of more than 200 ppm, especially more than 500 ppm, for example 600 to 2000 ppm.

Suitable TiO₂ materials are either obtained commercially or can be obtained by the person skilled in the art by standard methods, provided that it is ensured in the synthesis that the raw materials used comprise correspondingly low sulfur and calcium contaminations. Alternatively it is also possible to proceed from TiO₂ materials with a higher sulfur or calcium content and to establish contents suitable in accordance with the invention by suitable treatment, for example, leaching.

In a suitable embodiment, at least a portion of the titanium dioxide is treated with an aqueous medium under hydrothermal conditions. In the context of the present invention, hydrothermal conditions are understood to mean temperatures of at least 80° C. and pressures above atmospheric pressure (greater than 1 atm). Preference is given to temperatures between 120 and 500° C., particular preference to those between 180 and 300° C. and to pressures above atmospheric pressure, for example the autogenous pressure which is established at the given temperature in a closed vessel. The treatment under hydrothermal conditions may extend, for example, over 15 to 24 hours, preferably 30 min to 6 hours. A suitable aqueous medium is in particular water, for example demineralized or bidistilled water, or dilute acids or bases, such as 0.1-1 molar nitric acid, or 1 molar aqueous ammonia. Subsequently, the titanium dioxide material is removed from the aqueous medium, for example by filtration, and optionally washed and dried. The treatment can be repeated if desired. Typically, the titanium dioxide is used in the anatase form. The titanium dioxide preferably has a BET surface area of 15 to 60 m²/g, especially 15 to 45 m²/g, more preferably 13 to 28 m²/g. The titanium dioxide used may consist of a single titanium dioxide or a mixture of titanium dioxides. In the latter case, the value of the BET surface area is determined as the weighted mean of the contributions of the individual titanium dioxides. The titanium dioxide used consists, for example, advantageously of a mixture of a TiO₂ with a BET surface area of 5 to 15 m²/g and of a TiO₂ with a BET surface area of 15 to 50 m²/g.

The catalytically active material based on the total amount of the catalytically active material, preferably comprises 1 to 40% by weight of vanadium oxide, calculated as V₂O₅, and 60 to 99% by weight of titanium dioxide, calculated as TiO₂. The catalytically active material may, in preferred embodiments, additionally comprise up to 1% by weight of a cesium compound, calculated as Cs, up to 1% by weight of a phosphorus compound, calculated as P, and up to 10% by weight of antimony oxide, calculated as Sb₂O₃. All figures for the composition of the catalytically active material are based on the calcined state thereof, for example after calcination of the catalyst at 450° C. for 1 hour.

Suitable vanadium sources are particularly vanadium pentoxide or ammonium metavanadate.

Suitable antimony sources are various antimony oxides, especially antimony trioxide. In general, antimony trioxide with a mean particle size (maximum of the particle size distribution) of 0.1 to 10 μm is used. Particular preference is given to using, as the source of the antimony oxide in the first catalyst, particulate antimony trioxide with a mean particle size of 0.5 to 5 μm, especially 1 to 4 μm. Useful phosphorus sources include especially phosphoric acid, phosphorous acid, hypophosphorous acid, ammonium phosphate or phosphoric esters, and in particular ammonium dihydrogenphosphate. Useful sources of cesium include the oxides or hydroxide, or the salts which are convertible thermally to the oxide, such as carboxylates, especially the acetate, malonate or oxalate, carbonate, hydrogencarbonate, sulfate or nitrate.

In addition to the optional cesium and phosphorus additives, it is possible for a multitude of other oxidic compounds which, as promoters, influence the activity and selectivity of the catalyst, for example by lowering or increasing its activity, to be present in small amounts in the catalytically active material. Examples of such promoters include the alkali metal oxides, especially, apart from the cesium oxide mentioned, lithium oxide, potassium oxide and rubidium oxide, thallium(I) oxide, aluminum oxide, zirconium oxide, iron oxide, nickel oxide, cobalt oxide, manganese oxide, tin oxide, silver oxide, copper oxide, chromium oxide, molybdenum oxide, tungsten oxide, iridium oxide, tantalum oxide, niobium oxide, arsenic oxide, antimony oxide, cerium oxide.

In addition, among the promoters mentioned, useful additives are preferably also the oxides of niobium, and tungsten in amounts of 0.01 to 0.50% by weight, based on the catalytically active material.

The inert support material used may be virtually any prior art support material, as used advantageously in the preparation of eggshell catalysts for the oxidation of aromatic hydrocarbons to aldehydes, carboxylic acids and/or carboxylic anhydrides, for example quartz (SiO₂), porcelain, magnesium oxide, tin dioxide, silicon carbide, rutile, alumina (Al₂O₃), aluminum silicate, steatite (magnesium silicate), zirconium silicate, cerium silicate or mixtures of these support materials. The support material is generally nonporous. The expression “nonporous” is understood in the sense of “nonporous apart from technically inactive amounts of pores”, since a small number of pores may technically unavoidably be present in the support material, which ideally should not comprise any pores. Advantageous support materials which should be emphasized are especially steatite and silicon carbide. The form of the support material is generally not critical for the inventive precatalysts and eggshell catalysts. For example, it is possible to use catalyst supports in the form of spheres, rings, tablets, spirals, tubes, extrudates or spall. The dimensions of these catalyst supports correspond to those of catalyst supports typically used to prepare eggshell catalysts for the gas phase partial oxidation of aromatic hydrocarbons. Preference is given to using steatite in the form of spheres with a diameter of 3 to 6 mm or of rings with an external diameter of 5 to 9 mm and a length of 3 to 8 mm and a wall thickness of 1 to 2 mm.

The layer(s) of the eggshell catalyst are appropriately applied by spray application of a suspension of TiO₂ and V₂O₅, which optionally comprises sources of the abovementioned promoter elements, to the fluidized support. Before the coating, the suspension is preferably stirred for a sufficiently long period, e.g. 2 to 30 hours, especially 12 to 25 hours, in order to break up agglomerates of the suspended solids and to obtain a homogeneous suspension. The suspension typically has a solids content of 20 to 50% by weight. The suspension medium is generally aqueous, for example water itself or an aqueous mixture with a water-miscible organic solvent, such as methanol, ethanol, isopropanol, formamide and the like.

Generally added to the suspension are organic binders, preferably copolymers, advantageously in the form of an aqueous dispersion, of acrylic acid/maleic acid, vinylacetate/vinyllaurate, vinylacetate/acrylate, styrene/acrylate and vinylacetate/ethylene. The binders are commercially available as aqueous dispersions with a solids content of, for example 35 to 65% by weight. The amount of such binder dispersions used is generally 2 to 45% by weight, preferably 5 to 35% by weight, more preferably 7 to 20% by weight, based on the weight of the suspension.

The support is fluidized in, for example, a fluidized bed or moving bed apparatus in an ascending gas stream, especially air. The apparatus usually consists of a conical or spherical vessel in which the fluidizing gas is introduced from the bottom or from the top via an immersed tube. The suspension is sprayed via nozzles into the fluidized bed from the top, laterally or from the bottom. It is advantageous to use a riser tube arranged in the middle or concentrically around the immersed tube. Within the riser tube, there is a higher gas velocity which transports the support particles upward. In the outer ring, the gas velocity is only slightly above the fluidization velocity. Thus, the particles are moved vertically in circulation. A suitable moving bed apparatus is described, for example in DE-A 4006935.

In the course of coating of the catalyst support with the catalytically active material, coating temperatures of 20 to 500° C. are generally employed, in which case the coating can be effected under atmospheric pressure or under reduced pressure. In general, the coating is effected at 0° C. to 200° C., preferably at 20 to 150° C., especially at 60 to 120° C.

The catalytically active material can also be applied in two or more layers, in which case, for example, the inner layer has, or the inner layers have, an antimony oxide content of up to 15% by weight and the outer layer has an antimony oxide content reduced by 50 to 100%. In general, the inner layer of the catalyst contains phosphorus and the outer layer is low in phosphorus or phosphorus-free.

The layer thickness of the catalytically active material is generally 0.02 to 0.2 mm, preferably 0.05 to 0.15 mm. The active material content in the catalyst is typically 5 to 25% by weight, usually 7 to 15% by weight.

As a result of thermal treatment of the precatalysts thus obtained at temperatures of more than 200 to 500° C. the binder escapes by thermal decomposition and/or combustion from the layer applied. The thermal treatment is preferably effected in situ in the gas phase oxidation reactor.

The inventive catalysts are generally suitable for gas phase oxidation of aromatic C₆- to C₁₀-hydrocarbons, such as benzene, the xylenes, toluene, naphthalene or durene (1,2,4,5-tetramethylbenzene) to carboxylic acids and/or carboxylic anhydrides such as maleic anhydride, phthalic anhydride, benzoic acid and/or pyromellitic dianhydride.

One embodiment of the invention relates to a process for preparing phthalic anhydride, in which a gas stream which comprises molecular oxygen and o-xylene, naphthalene or mixtures thereof is contacted with an inventive catalyst.

For this purpose, the catalysts prepared in accordance with the invention are introduced into reaction tubes thermostated externally to the reaction temperature, for example by means of salt melts, and the reaction gas is passed over the catalyst bed thus prepared temperatures of generally 300 to 450° C., preferably of 320 to 420° C. and more preferably of 340 to 400° C., and at a pressure of generally 0.1 to 2.5 bar gauge, preferably of 0.3 to 1.5 bar gauge, with a space velocity of generally 750 to 5000 h⁻¹.

The reaction gas supplied to the catalyst is generally obtained by mixing a molecular oxygen-comprising gas which, apart from oxygen, may also comprise suitable reaction moderators and/or diluents, such as steam, carbon dioxide and/or nitrogen, with the aromatic hydrocarbon to be oxidized, in which case the molecular oxygen-comprising gas may comprise generally 1 to 100 mol %, preferably 2 to 50 mol % and more preferably 10 to 30 mol % of oxygen, 0 to 30 mol %, preferably 0 to 10 mol % of steam, and 0 to 50 mol %, preferably 0 to 1 mol % of carbon dioxide, remainder nitrogen. To obtain the reaction gas, the molecular oxygen-comprising gas is generally charged at 30 g to 150 g per m³ (STP) of gas of the aromatic hydrocarbon to be oxidized.

It has been found to be particularly advantageous when catalysts with different catalytic activities and/or different chemical compositions of their active materials are used in the catalyst bed. Preferably, in the case of use of two reaction zones, the catalyst present in the first reaction zone, i.e. that toward the gas inlet of the reaction gas, has a somewhat lower catalytic activity compared to the catalyst present in the second reaction zone, i.e. that toward the gas outlet. In general, the reaction is controlled by the adjustment of temperature such that the majority of the aromatic hydrocarbon present in the reaction gas is converted at maximum yield in the first zone. Preference is given to using three- to five-layer catalyst systems, especially three- and four-layer catalyst systems.

In a preferred embodiment of a three-layer catalyst system, the catalysts have the following composition:
for the first, uppermost layer (layer CL1):
   7 to 10% by weight of active material based on the overall catalyst, where this active material comprises:
   6 to 11% by weight of vanadium (calculated as V₂O₅)
   0 to 6% by weight of antimony trioxide
   0.1 to 1% by weight of an alkali metal (calculated as alkali metal), especially cesium oxide,
   and, as the remainder to 100% by weight, titanium dioxide in the anatase polymorph with a BET surface area of 10 to 25 m²/g;
for the second, middle layer (layer CL2):
   7 to 12% by weight of active material based on the overall catalyst, where this active material comprises:
   5 to 13% by weight of vanadium (calculated as V₂O₅)
   0 to 6% by weight of antimony trioxide
   0 to 0.4% by weight of an alkali metal (calculated as alkali metal), especially cesium oxide,
   0 to 0.4% by weight of phosphorus pentoxide (calculated as P)
   and, as the remainder to 100% by weight, titanium dioxide in the anatase polymorph with a BET surface area of 15 to 25 m²/g;
for the third, lowermost layer (layer CL3):
   8 to 12% by weight of active material based on the overall catalyst, where this active material comprises:
   5 to 30% by weight of vanadium (calculated as V₂O₅)
   0 to 6% by weight of antimony trioxide
   0 to 0.3% by weight of an alkali metal (calculated as alkali metal), especially cesium oxide,
   0.05 to 0.4% by weight of phosphorus pentoxide (calculated as P)
   and, as the remainder to 100% by weight, titanium dioxide in the anatase polymorph with a BET surface area of 15 to 30 m²/g.

In a preferred embodiment of a four-layer catalyst system, the catalysts have the following composition:
for the first layer (layer CL1):
   7 to 10% by weight of active material based on the overall catalyst, where this active material comprises:
   6 to 11% by weight of vanadium (calculated as V₂O₅)
   0 to 6% by weight of antimony trioxide
   0.1 to 1% by weight of an alkali metal (calculated as alkali metal), especially cesium oxide,
   and, as the remainder to 100% by weight, titanium dioxide in the anatase polymorph with a BET surface area of 5 to 20 m²/g;
for the second layer (layer CL2):
   7 to 12% by weight of active material based on the overall catalyst, where this active material comprises:
   4 to 15% by weight of vanadium (calculated as V₂O₅)
   0 to 6% by weight of antimony trioxide
   0.1 to 1% by weight of an alkali metal (calculated as alkali metal), especially cesium oxide,
   0 to 0.4% by weight of phosphorus pentoxide (calculated as P)
   and, as the remainder to 100% by weight, titanium dioxide in the anatase polymorph with a BET surface area of 10 to 25 m²/g;
for the third layer (layer CL3):
   7 to 12% by weight of active material based on the overall catalyst, where this active material comprises:
   5 to 15% by weight of vanadium (calculated as V₂O₅)
   0 to 6% by weight of antimony trioxide
   0 to 0.4% by weight of an alkali metal (calculated as alkali metal), especially cesium oxide,
   0 to 0.4% by weight of phosphorus pentoxide (calculated as P)
   and, as the remainder to 100% by weight, titanium dioxide in the anatase polymorph with a BET surface area of 15 to 25 m²/g;
for the fourth layer (layer CL4):
   8 to 12% by weight of active material based on the overall catalyst, where this active material comprises:
   5 to 30% by weight of vanadium (calculated as V₂O₅)
   0 to 6% by weight of antimony trioxide
   0.05 to 0.4% by weight of phosphorus pentoxide (calculated as P)
   and, as the remainder to 100% by weight, titanium dioxide in the anatase polymorph with a BET surface area of 15 to 30 m²/g.

In general, the catalyst layers CL1, CL2, CL3 and/or CL4 may also be arranged such that they each consist of two or more layers. These intermediate layers advantageously have intermediate catalyst compositions.

Instead of mutually delimited layers of the different catalysts, it is also possible to bring about a quasi-continuous transition of the layers and a quasi-homogeneous rise in the activity by implementing a zone with a mixture of the successive catalysts at the transition from one layer to the next layer.

The bed length of the first catalyst layer preferably makes up more than 30 to 80% of the total catalyst fill height in the reactor. The bed height of the first two or of the first three catalyst layers advantageously makes up more than 60 to 95% of the total catalyst fill height. Typical reactors have a fill height of 250 cm to 350 cm. The catalyst layers may also optionally be distributed between a plurality of reactors.

If desired, a downstream finishing reactor can also be provided for the phthalic anhydride preparation, as described, for example in DE-A 198 07 018 or DE-A 20 05 969. The catalyst used in this case is preferably an even more active catalyst compared to the catalyst of the last layer.

When the PA preparation is performed with the inventive catalysts using a plurality of reaction zones in which there are different catalysts, it is possible to use the novel eggshell catalysts in all reaction zones. However, it is generally already possible to achieve considerable advantages over conventional processes when inventive eggshell catalyst is used only in one of the reaction zones of the catalyst bed, for example, the first reaction zone, or the first two reaction zones, and eggshell catalysts produced in a conventional manner are utilized in the remaining reaction zones. In the first reaction zone(s), there are higher hotspot temperatures compared to the downstream reaction zones; the majority of the starting hydrocarbon is oxidized here to the desired oxidation product and/or intermediates, such that the advantages of the inventive catalysts are manifested particularly in the first stage or in the first and second stages. Preferably, inventive catalysts are used in at least 50% of the total bed length (in flow direction of the gaseous stream).

The invention is illustrated in detail by the examples which follow.

### Catalyst Preparation

The following titanium dioxides were used (all titanium dioxides were in the anatase polymorph):

| | BET surface | | | | |
|---|---|---|---|---|---|
| Desig- | area | S | Nb | P | Ca |
| nation | [m²/g] | [ppm] | [ppm] | [ppm] | [ppm] |
| A | 27 | 2400 | 1000 | 100 | 240 |
| B* | 25 | 360 | n.d. | 100 | 70 |
| C | 16 | 2400 | 1000 | 250 | 250 |
| D | 16 | 2400 | 1000 | 250 | 100 |
| E | 16 | 500 | 1000 | 250 | 250 |
| F | 16 | 300 | 1000 | 250 | 80 |
| G | 31 | 2600 | 1000 | 250 | 250 |
| H | 31 | 2600 | 1000 | 250 | 100 |
| I | 31 | 500 | 1000 | 250 | 250 |
| J | 31 | 900 | 1000 | 250 | 100 |

n.d. not determined

*Titanium dioxide B was prepared from titanium dioxide A by aftertreatment under hydrothermal conditions: 358 g of titanium dioxide A were suspended in 1099 g of water while stirring. This suspension was transferred to an autoclave and stirred at 370 rpm at 300° C. for 72 hours. The resulting suspension was filtered and the filtercake was washed with 2 liters of water and dried at 80° C. under pressure of less than 100 mbar for 16 hours. The sulfur and calcium contents of titanium dioxide B were significantly reduced by the pretreatment.

### Catalyst A (Non-Inventive)

1000 g of steatite spheres (diameter 3.5-4.5 mm) were coated in a fluidized bed coater with 14.3 g of organic binder (copolymer of acrylic acid and maleic acid, weight ratio W 75:25), and a suspension composed of 13.06 g of vanadium pentoxide, 34.79 g of oxalic acid, 5.64 g of antimony trioxide, 1.029 g of ammonium dihydrogenphosphate, 0.94 g of cesium sulfate, 175.93 g of water, 36.28 g of formamide and 113.38 g of titanium dioxide A. The active material thus prepared comprises an average of 0.21% by weight of phosphorus (calculated as P), 9.8% by weight of vanadium pentoxide (calculated as V₂O₅), 4.2% by weight of antimony trioxide (calculated as Sb₂O₃), 0.52% by weight of cesium (calculated as Cs) and 85.25% by weight of titanium dioxide (calculated as TiO₂). The coated catalyst thus prepared was subsequently coated with 14.4 g of organic binder (copolymer of acrylic acid and maleic acid, weight ratio=75:25) and a suspension composed of 6.97 g of vanadium pentoxide, 18.83 g of oxalic acid, 0.94 g of cesium sulfate, 178.4 g of water, 49.43 g of formamide and 125.34 g of titanium dioxide A. The second active material layer thus prepared comprises an average of 5.2% by weight of vanadium pentoxide (calculated as V₂O₅), 0.52% by weight of cesium (calculated as Cs) and 94.24% by weight of titanium dioxide (calculated as TiO₂). In total, a total active material content of 8.51% by weight was achieved with the two layers.

### Catalyst B (Inventive)

1000 g of steatite spheres (diameter 3.5-4.5 mm) were coated in a fluidized bed coater with 16.2 g of organic binder (copolymer of acrylic acid and maleic acid, weight ratio=75:25), and a suspension composed of 8.50 g of vanadium pentoxide, 22.64 g of oxalic acid, 3.68 g of antimony trioxide, 0.67 g of ammonium dihydrogenphosphate, 0.62 g of cesium sulfate, 121.33 g of water, 25.03 g of formamide and 78.22 g of titanium dioxide B. The active material thus prepared comprises an average of 0.20% by weight of phosphorus (calculated as P), 9.34% by weight of vanadium pentoxide (calculated as V₂O₅), 4.0% by weight of antimony trioxide (calculated as Sb₂O₃), 0.50% by weight of cesium (calculated as Cs) and 85.96% by weight of titanium dioxide (calculated as TiO₂). The coated catalyst thus prepared was subsequently coated with 17.8 g of organic binder (copolymer of acrylic acid and maleic acid, weight ratio=75:25) and a suspension composed of 4.58 g of vanadium pentoxide, 12.37 g of oxalic acid, 6.2 g of cesium sulfate, 122.73 g of water, 33.91 g of formamide and 85.97 g of titanium dioxide B. The second active material layer thus prepared comprises an average of 5.03% by weight of vanadium pentoxide (calculated as V₂O₅), 0.50% by weight of cesium (calculated as Cs) and 94.47% by weight of titanium dioxide (calculated as TiO₂). In total, a total active material content of 9.74% by weight was achieved with the two layers. The composition of the active materials for catalyst B was selected such that the same average (average of the two active material layers) number of vanadium pentoxide monolayers were applied in catalyst A and catalyst B. For this purpose 0.15% by weight of vanadium pentoxide was applied per m² of BET surface area of the titanium dioxide used.

Preparation of Catalyst Layer CL1CG (CL1 with Titanium Dioxides C and G, Bet Surface Area of the Titanium Dioxide Mixture=16.15 m²/g):

3.38 g of cesium carbonate, 649.6 g of titanium dioxide C, 6.58 g of titanium dioxide G, 51.37 g of vanadium pentoxide and 13.15 g of antimony trioxide were suspended in 1877 g of demineralized water and stirred for 18 hours, in order to achieve a homogeneous distribution. 77.7 g of organic binder (copolymer of vinylacetate and vinyllaurate in the form of a 50% by weight aqueous dispersion) were added to this suspension. In a moving bed apparatus, 660 g of this suspension were sprayed onto 2 kg of steatite (magnesium silicate) in the form of rings of dimensions 7 mm×7 mm×4 mm, and dried. After the catalyst had been calcined at 450° C. for one hour, the active material applied to the steatite rings was 9.1%. The analyzed composition of the active material consisted of 7.1% V₂O₅, 1.8% Sb₂O₃, 0.38% Cs, remainder TiO₂.

Catalyst CL2CG (Bet Surface Area of the Titanium Dioxide Mixture=18.25 m²/g):

The preparation was effected analogously to the preparation of CL1 with variation of the composition of the suspension. Titanium dioxide C and titanium dioxide G were used in a weight ratio of 85:15. After calcination of the catalysts at 450° C. for one hour, the active material applied to the steatite rings was 8.5%. The analyzed composition of the active material consisted of 7.95% V₂O₅, 2.7% Sb₂O₃, 0.31% Cs, remainder TiO₂.

Catalyst CL3CG (BET Surface Area of the Titanium Dioxide Mixture=16.75 m²/g):

The preparation was effected analogously to CL1 with variation of the composition of the suspension. Titanium dioxide C and titanium dioxide G were used in a weight ratio of 95:5. After calcination of the catalyst at 450° C. for one hour, the active material applied to the steatite rings was 8.5%. The analyzed composition of the active material consisted of 7.1% V₂O₅, 2.4% Sb₂O₃, 0.10% Cs, remainder TiO₂.

Catalyst CL4CG (BET Surface Area of the Titanium Dioxide Mixture=23.05 m²/g):

The preparation was effected analogously to CL1 with variation of the composition of the suspension. Titanium dioxide C and titanium dioxide G were used in a weight ratio of 53:47. After calcination of the catalyst at 450° C. for one hour, the active material applied to the steatite rings was 9.1%. The analyzed composition of the active material consisted of 20% V₂O₅, 0.38% P, remainder TiO₂.

Further catalyst layers were prepared by the above-described method, except that different titanium dioxides were used. The nomenclature of the catalyst layers follows the scheme described above. Catalyst layer CL3DH was a catalyst layer with the composition of CL3, in which, however, titanium dioxides D and H had been used.

### COMPARATIVE EXAMPLE 1

Catalyst testing in a screening reactor: an iron tube of length 80 cm with an internal width of 15 mm was charged with 66 cm of catalyst A. The tube was surrounded by a salt melt for temperature regulation. 360 l (STP)/h of air per hour were passed through the tube from the top downward with loadings of 56 g of o-xylene/m³ (STP) of air in the form of 98.5% by weight o-xylene. At a reactor temperature of 350° C., a PA yield of 75.2 mol % was achieved (the “PA yield” means the phthalic anhydride obtained in mole percent or percent by weight, based on 100% o-xylene).

### EXAMPLE 2

Comparative example 1 was repeated, except that 66 cm of catalyst B were introduced into the iron tube. At a reactor temperature of 343° C. and a loading of 56 g/m³ (STP), a PA yield of 80.1 mol % was achieved.

### COMPARATIVE EXAMPLES 3 TO 5 AND EXAMPLE 6

Catalyst testing in a model tubular reactor: the catalytic oxidation of o-xylene to phthalic anhydride was performed in a salt bath-cooled tubular reactor with an internal diameter of the tubes of 25 mm. From the reactor inlet to reactor outlet, 130 cm of CL1, 70 cm of CL2, 60 cm of CL3 and 60 cm of CL4 were introduced into an iron tube of length 3.5 m with an internal width of 25 mm. The iron tube was surrounded by a salt melt for temperature regulation; a thermowell of external diameter 4 mm with an installed thermocouple served to measure the catalyst temperature. 4.0 m³ (STP) of air per hour were passed through the tube from the top downward with loadings of 99.2% by weight o-xylene of 30 to 100 g/m³ (STP).

| | | Salt bath | o-xylene | PA |
|---|---|---|---|---|
| | | temper- | loading | yield |
| Exam- | | ature | [g/m³ | [% by |
| ple | Catalyst layers | [° C.] | (STP)] | wt.] |
| 3 | CL1CG, CL2CG, CL3CG, CL4CG | 361 | 66 | 111.2 |
| 4 | CL1DH, CL2DH, CL3DH, CL4DH | 361 | 66 | 113.0 |
| 5 | CL1EI, CL2EI, CL3EI, CL4EI | 361 | 66 | 112.6 |
| 6 | CL1FJ, CL2FJ, CL3FJ, CL4FJ | 361 | 66 | 114.1 |

It is found that, as well as the sulfur content, the calcium content of the titanium dioxide used greatly influences the PA yield.

1.-11. (canceled)

12. A catalyst for gas phase oxidation comprising an inert support and a catalytically active material which comprises vanadium oxide and titanium dioxide and has been applied thereto, wherein the titanium dioxide has a content of sulfur compounds, calculated as S, of less than 1000 ppm and a content of calcium compounds, calculated as Ca, of less than 150 ppm.

13. The catalyst according to claim 12, wherein the titanium dioxide has a content of sulfur compounds, calculated as S, of less than 500 ppm.

14. The catalyst according to claim 12, wherein the titanium dioxide has a content of calcium compounds, calculated as Ca, of less than 100 ppm.

15. The catalyst according to claim 12, wherein the titanium dioxide has a BET surface area of 15 to 60 m²/g.

16. The catalyst according to claim 13, wherein the titanium dioxide has a content of calcium compounds, calculated as Ca, of less than 100 ppm and the titanium dioxide has a BET surface area of 15 to 60 m²/g.

17. The catalyst according to claim 12, wherein at least a portion of the titanium dioxide has been treated with an aqueous medium under hydrothermal conditions.

18. The catalyst according to claim 12, wherein the catalytically active material comprises 1 to 40% by weight of vanadium oxide, calculated as V₂O₅, and 60 to 99% by weight of titanium dioxide, calculated as TiO₂.

19. The catalyst according to claim 18, wherein the catalytically active material comprises up to 1% by weight of a cesium compound, calculated as Cs, up to 1% by weight of a phosphorus compound, calculated as P and up to 10% by weight of antimony oxide, calculated as Sb₂O₃.

20. A process for preparing a catalyst for gas phase oxidations which comprises applying a suspension of titanium dioxide and vanadium oxide particles to an inert support, wherein the titanium dioxide has a content of sulfur compounds, calculated as S, of less than 1000 ppm and a content of calcium compounds, calculated as Ca, of less than 150 ppm.

21. The process according to claim 20, wherein the suspension also comprises at least one cesium, phosphorus and/or antimony source.

22. The process according to claim 20, wherein the application of at least a portion of the titanium dioxide is preceded by treatment with an aqueous medium under hydrothermal conditions.

23. A process for preparing phthalic anhydride, which comprises contacting a gas stream which comprises molecular oxygen and o-xylene, naphthalene or mixtures thereof with the catalyst according to claim 12.
